(19) United States
(12) Patent Application Publication
(10) Pub. No.: US 2009/0198223 A1
(43) Pub. Date: Aug. 6, 2009

(54) SYSTEMS AND METHODS FOR CARDIAC ABLATION USING LASER INDUCED OPTICAL BREAKDOWN

(75) Inventors: Rachel Estelle Thilwind, Utrecht (NL); Robbert Adrianus Maria van Hal, St. Oedenrode (NL); Rieko Verhagen, Vught (NL); Paul Anton Josef Ackermans, Nuenen (NL); Dirk Brokken, Nuenen (NL); Jozef J.M. Janssen, Stevensweert (NL); Bart Gottenbos, Budel (NL); Sieglinde Neerken, Eindhoven (NL); Francisco Morales Serrano, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/297,012

(22) PCT Filed: Apr. 2, 2007
(86) PCT No.: PCT/IB07/51177

§ 371 Date: Oct. 13, 2008

(51) Int. Cl. A61B 18/24; A61B 5/04

(52) U.S. Cl. 606/15; 600/509

(57) ABSTRACT

Systems and methods for achieving sub-surface, highly spatially selective cardiac ablation by means of laser induced optical breakdown (LIOB) are disclosed. Damage to non-targeted heart and artery/vein tissue is to be minimized according to the present disclosure. A catheter enters the heart, e.g., via a vein, and catheter location is determined/confirmed. Laser pulses are guided through the optical path within the catheter and, at or near the catheter end, a focusing structure is provided that focuses the laser radiation through the non-targeted vein/heart tissue into the targeted tissue. In the focusing structure, laser induced LIOB occurs and related mechanical effects affect the targeted tissue.

The present disclosure is directed to systems and methods for achieving spatially selective tissue ablation and, more particularly, to systems and methods for undertaking spatially selective cardiac ablation by means of laser induced optical breakdown (LIOB). The disclosed systems and methods are adapted for in-vivo clinical applications and may be implemented with respect to organs/structures below the tissue surface.

Atrial fibrillation and other cardiac arrhythmias present major challenges to the medical profession, especially in the developed world, where prevalence of such ailments increases with age. These conditions are generally manifested by a rapid heart rate, dizziness, shortness of breath, pain, and lack of physical endurance, and can increase an individual's susceptibility to serious diseases of the heart, such as ischemia, stroke and heart failure.

In a normal mammalian heart, the atrial and ventricular muscles contract according to a synchronized excitation that originates from an action potential generated in the sino-atrial (SA) node (found in the wall of the right atrium). The action potential propagates along orderly conductive paths in the atrium to the atrioventricular (AV) node, causing the atrium to contract. From the AV node, the action potential then propagates through the bundle of His-Purkinje where it causes contraction of the ventricle.

The underlying cause of atrial fibrillation is a pathological condition of the cardiac tissue that leads to the disorderly conduction of asynchronous eddies of electrical impulses, which scatter about the atrial chamber and initiate an elevation of the heart beat rate and either paroxysmal or chronic tachycardia. Conventional treatments include pharmacological and surgical intervention, both of which can cause significant side effects. Patients who do not respond well to medication may be candidates for an implanted defibrillator device, or a surgical procedure known as Cox-Maze. This procedure involves the creation of several incisions in the atrial wall, followed by suturing, to create a maze-like pattern that blocks the conduction of the asynchronous impulses causing atrial fibrillation. In addition, the ostia (openings) of the pulmonary veins into the left atrium are sometimes electrically isolated during the Cox-Maze procedure, as there is evidence to suggest that a large proportion of the asynchronous impulses originate there. The technique is practiced only by very highly trained individuals, and requires long periods of theater/surgical time.

Ablation technologies and/or treatments have been developed that are intended to emulate the Cox-Maze procedure. For example, a catheter may be introduced into the atrium to thermally induce necrosis (at about 60° Celsius) of the myocardial tissue at selected locations. The necrosis causes the formation of scar tissue and thereby a conductive block to the asynchronous impulses, as achieved by the Cox-Maze. However, it is important to note that tissue should not be removed or perforated, as may be implied. Several different energy sources have been employed in such catheter-based ablation systems, the most popular energy sources being radio-frequency (RF) and cryothermal. More recently, ultrasound, microwave, and laser energy sources have received increasing interest as alternatives to RF and cryothermal.

In standard RF ablation, resistive heating occurs and the catheter can in theory create significant scar tissue, e.g., scar tissue that is up to 5 mm in diameter and 3 mm deep, but the effects are limited by the conditions within the heart and especially the cooling effect of the blood flowing in the atrium. The surface tissue is often adversely affected by the application of RF, with results such as charring and undesirable adhesion of the catheter to the tissue that then causes insulation and reduces efficient application of the energy. If the scars are not transmural (i.e., do not penetrate the full thickness of the myocardium), then full conduction block cannot be guaranteed, and the thickness of the atrial wall may vary significantly, e.g., ten-fold, within one ablation line. Therefore, control of ablation depth is crucial in the effectiveness of the procedure. In addition, Thomas et al. (‘Production of Narrow but Deep Lesions . . . ,’ Las. Surg. Med. 38:375-380 (2001)) states that lines of RF ablation are broad and that the loss of atrial mass may impair function and lead to an increased risk of stroke.

On the contrary, Thomas describes scars created by a laser catheter to be deeper and narrower. Fried et al. (‘Linear lesions in heart tissue using diffused laser radiation,’ Lasers in Surgery, Proc. SPIE Vol. 3907 (2000)) also implicates laser as a more appropriate energy source in ablation of atrial tissue, describing the potential for deeper tissue heating and a reduced risk of surface coagulation (which can lead to thromboembolic events) and vaporization.

State of the art laser catheter systems for cardiac ablation include optical fiber-based devices with radial diffusive tips, as well as those that deliver on-axis radiation. The majority operate at wavelengths in the near infra-red or infra-red range (typically 980 nm or 1064 nm), with delivery of between 20 and 80 W of power. Balloons have been designed to guide and distribute the energy in such a way as to encircle the ostia of the pulmonary veins. However, there are still problems surrounding this concept due to risks of pulmonary vein stenosis (closing).

With reference to the patent literature, several patent-related publications are noted. U.S. Patent Publication No. 2005/0165391 A1 to Maguire et al. discloses a tissue ablation device/assembly and method for electrically isolating a pulmonary vein ostium from an atrial wall. The Maguire tissue ablation system treats atrial arrhythmia by ablating a circumferential region of tissue at a location where a pulmonary vein extends from an atrium using a circumferential ablation member with an ablation element. The circumferential ablation member is generally adjustable between different configurations to allow both the delivery through a delivery sheath into the atrium and ablative coupling between the ablation element and the circumferential region of tissue.

U.S. Patent Publication No. 2005/0143722 A1 to Brucker et al. discloses a laser-based maze procedure for atrial fibrillation. A lesion formation tool is positioned against an accessed surface according to the Brucker publication. The tool includes an optical fiber for guiding a coherent waveform of a selected wavelength to a fiber tip for discharge of light energy from the fiber tip. The wavelength is selected for the light energy to penetrate a full thickness of the tissue to form a volume of necrosed tissue through the thickness of the tissue. The tool further includes a guide tip coupled to the fiber tip, the guide tip being adapted to have a discharge bore aligned with the fiber tip to define an unobstructed light pathway from the fiber tip to the tissue surface. The guide tip may be placed against the tissue surface with the guide tip slidable along the tissue surface. The Brucker lesion formation tool is intended to be manipulated so as to draw the guide tip over the tissue surface in a pathway while maintaining the discharge bore opposing the tissue surface to form a transmural lesion in the tissue extending a length of the pathway.

U.S. Pat. No. 6,893,432 B2 to Intintoli et al. discloses a light-dispersive probe that disperses light sideways from its fore end. A light-dispersive and light-transmissive medium is enclosed within a housing. The medium is divided into sections containing different concentrations of a light-dispersing material within a matrix, the sections being separated by non-dispersive spacers. At the tip end of the probe is a mirror to reflect the light back into the dispersive medium. By these features, the directionality and intensity distribution of the emitted light may be controlled.

U.S. 2005/0182393 A1 to Abboud et al. discloses a multi-energy ablation station that allows for a variety of ablation procedures to be performed without the interchanging of catheters. A console is provided that is connected to one or more energy treatment devices, such as catheters or probes, via an energy-delivering umbilical system. A processor in the console allows a user to selectively control which type of energy is released into the umbilical system and delivered to the energy treatment devices. Cryogenic fluid, RF energy, microwave or direct current, as well as laser energy can be supplied in order to cover a wide range of ablation techniques. The integrated ablation station is compatible with commercial catheters and allows for sequential or simultaneous ablation and mapping procedures to be performed when a deeper and wider lesion capability and/or a broader temperature ablation spectrum is desired.

U.S. Patent Publication No. 2005/0171520 A1 to Farr et al. is directed to a phototherapeutic wave guide apparatus for forming annular lesions in tissue. The optical apparatus disclosed in the Farr publication includes a pattern-forming optical wave guide in communication with a light transmitting optical fiber. Energy is transmitted through the optical fiber, such that radiation is propagated through the optical fiber and the wave guide projects an annular light pattern, e.g., a circle or a halo, onto tissue.

Additional patent literature of background interest includes PCT Publication WO 0311160 A2, which describes a cooled laser catheter for ablation of cardiac arrhythmias. The catheter limits damage to surface tissue while coagulating tissue within the myocardium. Lesions originate on average at 1 mm below the endocardial surface. The catheter can also include means for electro-physiological mapping of the heart. U.S. Pat. No. 5,836,941 to Yoshihara et al. describes a laser probe for treating hypertrophied prostate tissue. The laser beam can focus within the body tissue, and U.S. Pat. No. 5,651,786 to Abela et al. describes a mapping catheter having a laser. The catheter can localize a ventricular arrhythmia focus and destroy it by applying laser energy.

Turning specifically to laser-based technologies, lasers allow light to interact with materials in nanosecond/femtosecond period(s), with peak powers many orders of magnitude higher than that of continuous wave light but with low average powers. Interestingly, an optically transparent material that has no linear absorption of incident laser light may have strong non-linear absorption under high intensity irradiation of a femtosecond pulsed laser. This non-linear absorption can lead to photodisruption of the material by generating a fast, expanding high-temperature plasma. See, e.g., “Laser-induced breakdown in aqueous media,” Paul K Kennedy, Daniel X Hammer, Benjamin A Rockwell, Prog. Quant. Electr. 21:3:155-248 (1997); “Laser induced plasma formation in water at nanosecond to femtosecond time scales: Calculation of thresholds, absorption coefficients and energy density,” Joachim Noack, Alfred Vogel, IEEE Journal of Quantum Electronics, 38:8 (1999).

Measurable secondary effects of the plasma include shock wave emission, temperature increases, and cavitation bubble generation. Many applications of laser-induced optical breakdown (LIOB) have been developed recently, such as micromachining of solid materials, microsurgery of tissues, and high-density optical data storage. LIOB occurs when sufficiently high threshold intensity is attained at the laser focus, inducing plasma formation. Plasma formation leads to non-linear energy absorption and measurable secondary effects that include shock-wave emission, heat transfer, and cavitation bubbles (i.e., photodisruption). The presence and magnitude of these breakdown attributes are used to determine a material's LIOB threshold. Accordingly, the parameters applied in the generation of LIOB can generally be engineered to suit the properties of specific material(s). LIOB with nanosecond/femtosecond pulsed lasers is utilized in diverse applications, including biomedical systems, material characterization, and data storage.

Despite efforts to date, a need remains for systems and methods that are effective for achieving spatially selective tissue ablation. In addition, a need remains for systems and methods that can ablate at precise locations to a desired depth according to clinically relevant parameters. Still further, a need remains for systems and methods having particular applicability to cardiac ablation and that are effective to ablate to a desired depth according to the thickness of the myocardium and with a controlled geometry. Further, a need remains for systems and methods that are effective to achieve desired levels of cardiac ablation, while minimizing and/or eliminating potential damage to non-targeted heart and artery/vein tissue. These and other needs are satisfied by the disclosed systems and methods, as described herein.

The disclosed systems and methods are advantageously adapted to deliver spatially selective tissue ablation. According to exemplary embodiments, spatially selective cardiac ablation is delivered by means of laser induced optical breakdown (LIOB). The disclosed systems and methods are adapted for in-vivo clinical applications and may be implemented with respect to organs/structures below the tissue surface. In addition, potential damage to non-targeted heart and artery/vein tissue is minimized.

In exemplary embodiments of the present disclosure, a catheter is introduced to the heart, nearby and/or adjacent to the tissue to be treated. An optical path is defined within the catheter, e.g., one or more fiber optics. Generally, a vein is used as a gateway to the heart, although alternative minimally invasive techniques may be employed. Detection means are generally employed to determine the exact location for the treatment, e.g., conventional non-invasive imaging techniques. Once positioned in a desired clinical location, laser pulses are guided through the optical path within the catheter. At or adjacent the catheter end, a focus means functions to focus the laser radiation through the intermediate, non-targeted vein, heart and/or other tissue into the targeted tissue. Exemplary focus means include adaptive focusing structures/mechanisms, fluid focus lens systems, fixed focusing structures such as lenses and/or mirrors, and combinations thereof. In the focus region, LIOB occurs and the mechanical effects, e.g., shock waves, generated by such LIOB advantageously affect desired levels of ablation with respect to the targeted tissue.

As heart and vein tissue can both be regarded as being turbid media, with similar optical properties in the near infra-red (NIR) region, it is possible to execute highly spatial selective cardiac ablation below the tissue surface without damaging the surface tissue itself. Through control and/or modification of laser-related parameters, e.g., pulse energy, pulse duration and the like, clinicians can exercise a level of control over the operation of the disclosed system/method to achieve desired ablation results.

Additional features, functions and advantages associated with the disclosed systems and methods will be apparent from the description which follows, particularly when read in conjunction with the accompanying figures.

To assist those of ordinary skill in the art in making and using the disclosed systems and methods, reference is made to the accompanying figures, wherein:

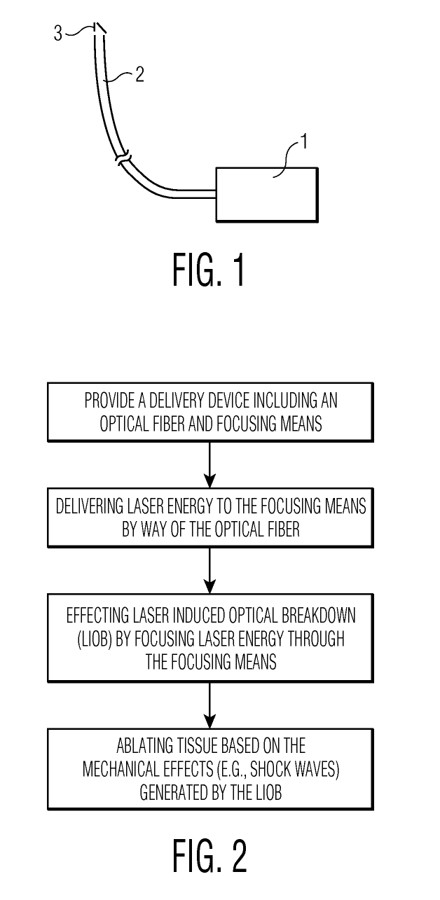

FIG. 1 provides a schematic illustration of an exemplary system according to the present disclosure; and

FIG. 2 provides a flowchart for an exemplary treatment method according to the present disclosure.

The disclosed systems and methods deliver spatially selective tissue ablation to target tissue. Spatially selective cardiac ablation is achieved, at least in part, through laser induced optical breakdown (LIOB) and the mechanical effects generated thereby. The disclosed systems and methods are adapted for in-vivo clinical applications, e.g., catheter-based clinical procedures, and may be implemented with respect to organs/structures below the tissue surface. Potential damage to non-targeted heart and artery/vein tissue is advantageously minimized through the LIOB-based ablation techniques and systems of the present disclosure.

In general, the systems and methods of the present disclosure are adapted to generate and deliver strongly focused, short-pulsed, laser pulses to the clinical region of interest. The laser pulses are advantageously generated/delivered at a wavelength that is minimally absorbed and scattered by heart and vein tissue, and is focused through the non-targeted tissue into/onto the tissue to be treated. When the electrical fields associated with the laser focus are strong enough to ionize material very locally, optical breakdown occurs and the associated mechanical effects (e.g., shock waves) cause a well-confined damage array around the focal area. The impact range of the mechanical effects can be engineered/controlled by adjusting the laser parameters (e.g., pulse energy and pulse duration).

Techniques and parameters for effecting LIOB according to the present disclosure are generally selected to achieve desired clinical results. More particularly, a variety of wavelengths, pulse times, power densities and related operating parameters may be employed to effect the desired mechanical effects based on the LIOB phenomenon. Indeed, operating parameters in the ranges described in a commonly assigned PCT patent publication entitled “A Device for Shortening Hairs by Means of Laser Induced Optical Breakdown Effects” to Van Hal et al. (WO 2005/011510 A1), have been found to be effective in achieving the desired LIOB effect for purposes of cardiac ablation, as described herein. The entire contents of the foregoing PCT publication are hereby incorporated herein by reference.

According to exemplary embodiments of the present disclosure, it is possible to treat a target area in a single focus, single pulse mode. However, in alternative embodiments, subsequently applied pulses and/or simultaneously generated foci may be delivered to the clinical region of interest. The subsequently applied pulses and/or simultaneously generated foci generally result in a similar number of simultaneously occurring LIOB centers inside the target area.

In an exemplary embodiment of the present disclosure and with reference to FIG. 1, a delivery device is provided that contains a length of laser-coupled optical fiber and focusing means (3) to direct energy to induce optical breakdown of tissue at a location defined by intra-cardial mapping. The laser energy source (1) is sufficient means to produce energy that, when directed at cardiac tissue, will induce optical breakdown in that tissue. The optical fiber delivery system (2) includes single or multiple optical fibers, photonic crystal fibers, fiber lasers and/or combinations thereof, and is generally compatible with balloon-shaped optical guides and/or other conventional catheter technologies.

According to an exemplary embodiment of the disclosed systems/methods, the mapping tool (visible during fluoroscopy) for measuring electrical stimuli within the cardiac tissue is integrated into the delivery device. Alternatively, the mapping tool may be associated with a separate probe. The mapping tool may take a variety of forms, but in an exemplary embodiment, such mapping tool includes a quadripolar probe. When inserted and removed from the pulmonary vein into the left atrium, the quadripolar probe is adapted to register a sudden decrease in impedance in conjunction with the presence of atrial potential (see, e.g., ‘Atrial Electroanatomic Remodelling . . . ,’ Pappone et al, Circulation 2001:104:2539-2544).

From a clinical standpoint, the delivery device, e.g., a catheter, can be inserted into a blood vessel in the neck or groin for access to the endocardium, and can be utilized during both minimally-invasive or by-pass surgery. The delivery device can also be applied to the epicardium. The optical fiber delivery system is generally compatible with MRI, X-ray fluoroscopy and other imaging modalities, thereby facilitating positioning of the catheter and associated LIOB-based focus means with respect to the tissue of interest. Elements used for measuring electrical, optical or mechanical changes to the substrate during ablation, e.g., piezoelements, optical or electrical sensors, and/or combinations thereof, can be incorporated in the delivery device. Moreover, the delivery device can be controlled by means of an adjustable control system, such that the energy delivered to the region of interest can be adapted to suit the energy requirements defined, in whole or in part, by a mapping device.

With reference to FIG. 2, an exemplary method/technique for achieving highly spatially selective cardiac ablation according to the present disclosure is provided. A delivery device is provided, e.g., a catheter, for transmitting laser energy to a region of interest. As noted previously, the delivery device/catheter may be adapted to cooperate/interact with conventional catheter technologies, e.g., guidewires, balloon-shaped guides and the like. In addition, the delivery device/catheter is advantageously adapted to facilitate minimally-invasive positioning and position-monitoring, e.g., through conventional imaging techniques such as fluoroscopy.

The delivery device/catheter includes or is adapted to receive an optical fiber element(s), e.g., through a lumen positioned therewithin. The optical fiber is adapted to be coupled to a laser source at one end (i.e., the proximal end), and optically communicates with a focusing means at the opposite end (i.e., the distal end). The laser source is adapted to generate and deliver appropriate energy pulses, as described herein. The clinician is generally permitted to control and/or select laser operating parameters, e.g., pulse energy and pulse duration, although preset operating conditions may also be provided with respect to the laser system, thereby reducing the potential for operator error. The focusing means is advantageously adapted to focus/direct the laser energy so as to induce optical breakdown of tissue at a location defined by intra-cardial mapping.

The laser induced optical breakdown induced by the focused energy is effective to ablate tissue, at least in part based on the mechanical effects, e.g., shock waves, generated by such LIOB. The focusing means according to the present disclosure may take a variety of forms, e.g., adaptive focusing structures/mechanisms, fluid focus lens systems, fixed focusing structures such as lenses and/or mirrors, and combinations thereof. Ablation according to the disclosed method/technique advantageously effects little or no damage to surrounding tissue, e.g., non-targeted heart tissue and artery/vein tissue.

Thus, the present disclosure provides advantageous systems and methods for achieving subsurface, highly spatially selective cardiac ablation by means of laser induced optical breakdown (LIOB) in-vivo. Although the present disclosure has been described with reference to exemplary embodiments and implementations thereof, the present disclosure is not limited to or by such exemplary embodiments. Rather, the present disclosure may be further enhanced, modified and/or altered based on the description provided herein without departing from the spirit or scope hereof Accordingly, the present disclosure expressly encompasses within its scope any and all such enhancements, modifications and/or alterations.

1. A method for cardiac ablation, comprising:
a. providing a delivery device that is configured and dimensioned for positioning adjacent a region of interest, the delivery device including an optical fiber and a focusing means;
b. delivering laser energy through the optical fiber to the focusing means;
c. effecting laser induced optical breakdown (LIOB) by focusing the laser energy through the focusing means so as to induce optical breakdown of tissue; and
d. ablating target tissue based on mechanical effects of the LIOB.
e.

2. A method according to claim 1, wherein the LIOB is effective in ablating target tissue that is below surface tissue, and wherein the surface tissue is left substantially intact.

3. A method according to claim 1, wherein the surface tissue defines non-target tissue.

4. A method according to claim 1, wherein the LIOB is effective in inducing optical breakdown of tissue in a highly localized focus region.

5. A method according to claim 1, wherein the ablation of target tissue is achieved without substantial damage to non-target tissue and/or artery/vein tissue.

6. A method according to claim 1, wherein the delivery device is or includes a catheter.

7. A method according to claim 1, wherein the delivery device includes one or more elements for measuring electrical, optical or mechanical changes to the tissue during ablation, e.g., piezoelement(s), optical or electrical sensor(s), and/or combinations thereof.

8. A method according to claim 1, wherein the delivery device includes an optical fiber delivery system.

9. A method according to claim 8, wherein the optical fiber delivery system includes single or multiple optical fibers, photonic crystal fibers, fiber lasers and/or combinations thereof.

10. A method according to claim 1, wherein the region of interest is cardiac tissue.

11. A method according to claim 1, wherein the LIOB is effected in a region selected through intra-cardial mapping.

12. A method according to claim 1, wherein the delivery device is compatible with imaging technology, e.g., MRI, X-ray, fluoroscopy and the like.

13. A method according to claim 1, further comprising a mapping tool for measuring electrical stimuli within the cardiac tissue.

14. A method according to claim 13, wherein the mapping tool is integrated into the delivery device.

15. A method according to claim 13, wherein the mapping tool is an independent structure relative to the delivery device.

16. A method according to claim 13, wherein the mapping tool includes a quadripolar probe which is adapted to register a sudden decrease in impedance in conjunction with the presence of atrial potential.

17. A system adapted to perform the method as claimed in claim 1.
